(19) United States
(12) Patent Application Publication
(10) Pub. No.: US 2015/0157362 A1
(43) Pub. Date: Jun. 11, 2015

(54) SPINAL IMPLANT SYSTEM AND METHOD

(71) Applicant: Warsaw Orthopedic, Inc., Warsaw, IN (US)

(72) Inventors: Kevin T. Foley, Germantown, TN (US); Roy Lim, Germantown, TN (US); Newton H. Metcalf, Memphis, TN (US)

(73) Assignee: Warsaw Orthopedic, Inc., Warsaw, IN (US)

(21) Appl. No.: 14/098,065

(22) Filed: Dec. 5, 2013

(51) Int. Cl. A61B 17/70

(52) U.S. Cl. CPC A61B 17/70

(57) ABSTRACT

A spinal construct includes a guide having a first member and a second member. The first member includes an inner surface defining a guide cavity. The second member extends in a transverse orientation relative to the first member. A first fastener includes a first end and a second end. The first end defines an implant cavity and a second end is configured for penetrating tissue. A second fastener is disposable in the guide cavity such that the inner surface aligns the second fastener for penetrating tissue. Systems and methods are disclosed.

### TECHNICAL HELD

The present disclosure generally relates to medical devices for the treatment of musculoskeletal disorders, and more particularly to a surgical system for implant delivery to a surgical site and a method for treating a spine.

### BACKGROUND

Spinal pathologies and disorders such as scoliosis and other curvature abnormalities, kyphosis, degenerative disc disease, disc herniation, osteoporosis, spondylolisthesis, stenosis, tumor, and fracture may result from factors including trauma, disease and degenerative conditions caused by injury and aging. Spinal disorders typically result in symptoms including deformity, pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes fusion, fixation, correction, discectomy, laminectomy and implantable prosthetics. As part of these surgical treatments, spinal constructs such as bone fasteners and vertebral rods are often used to provide stability to a treated region. For example, during surgical treatment, surgical instruments can be used to prepare a surgical site and bone fasteners can be delivered to the surgical site for fixation with bone to immobilize a joint. This disclosure describes an improvement over these prior art technologies.

### SUMMARY

In one embodiment, a spinal construct is provided. The spinal construct includes a guide having a first member and a second member. The first member includes an inner surface defining a guide cavity. The second member extends in a transverse orientation relative to the first member. A first fastener includes a first end and a second end. The first end defines an implant cavity and a second end is configured for penetrating tissue. A second fastener is disposable in the guide cavity such that the inner surface aligns the second fastener for penetrating tissue. In some embodiments, systems and methods are disclosed.

### BRIEF DESCRIPTION OF THE DRAWINGS

The present disclosure will become more readily apparent from the specific description accompanied by the following drawings, in which:

FIG. 1 is a perspective view of components of one embodiment of a system in accordance with the principles of the present disclosure with parts separated;

FIG. 2 is a perspective view of components of one embodiment of a system in accordance with the principles of the present disclosure disposed with vertebrae;

FIG. 3 is a perspective view of components of the system and the vertebrae shown in FIG. 2;

FIG. 4 is a plan view of components of the system and the vertebrae shown in FIG. 2;

FIG. 5 is a plan view of components of the system and the vertebrae shown in FIG. 2;

FIG. 6 is a perspective view of components of the system and the vertebrae shown in FIG. 2;

FIG. 7 is a side view of components of the system and the vertebrae shown in FIG. 2;

FIG. 8 is a perspective view of components of the system and the vertebrae shown in FIG. 2;

FIG. 9 is a perspective view of components of the system and the vertebrae shown in FIG. 2;

FIG. 10 is a perspective view of components of the system and the vertebrae shown in FIG. 2;

FIG. 11 is a plan view of components of the system and the vertebrae shown in FIG. 2; and

FIG. 12 is a perspective view of components of the system and the vertebrae shown in FIG. 2.

### DETAILED DESCRIPTION

The exemplary embodiments of the surgical system and related methods of use disclosed are discussed in terms of medical devices for the treatment of musculoskeletal disorders and more particularly, in terms of a surgical system for implant delivery to a surgical site and a method for treating a spine. In one embodiment, the systems and methods of the present disclosure are employed with a spinal joint and fusion, for example, with a cervical, thoracic, and/or lumbar region of a spine. In one embodiment, the systems and methods can be employed with a spinal fusion, such as, for example, a posterior joint fusion. In one embodiment, the components of the present system are employed for an atlanto-axial fusion. In one embodiment, the components of the present system are employed for stabilizing the C1 and C2 cervical vertebrae through fixation.

In one embodiment, the system includes a cervical guide tube and spinal rod implant device. In one embodiment, the system includes a tube and rod implant and a lag screw. In one embodiment, the device includes a drill and/or tap and/or screw guide and resists and/or prevents cervical nerve root damage and vertebral artery injury during implantation. In one embodiment, the system includes placement of a tube of the device docked onto a cervical lateral mass and a multi-axial screw (MAS) in the pars interarticularispars of a cervical vertebral body. In one embodiment, the system includes fixation of MAS with the cervical lateral mass in parallel and such that the entry point of the tubes are away from the anatomy that is at risk during drilling and tapping. In one embodiment, the device includes an implant tube positioned such that the cervical exiting nerve root can be preserved and protected during drilling and/or tapping and/or screw placement. This configuration provides space between a cervical exiting nerve root and the implant tube.

In one embodiment, the system is employed with a method for implanting the components of the system with a lateral side of vertebrae. In one embodiment, the method comprises the steps of initially placing MAS into the pars of the C2 vertebral body; aligning the implants with the C1 lateral masses and docking the tubes with the C2 pars MAS; verifying with the tube that the implant orientation selected is satisfactory; verifying that the bilateral tube implants are in position and locked into place by the MAS; and drilling and/or tapping the C1 lateral masses safely and the lag screws implanted. In one embodiment, the system is implanted with a contra-lateral side of the vertebrae. In one embodiment, the system includes graft implanted between the C1 arch and C2 spinous process. In one embodiment, the system includes a crosslink configured for connection with the implants.

In one embodiment, the systems may be employed with a method that comprises the steps of determining joint trajectory visually for example in an open surgical procedure, or through the use of a fluoroscope or image guidance such as, for example, an O-arm and/or surgical navigation. In one embodiment, the method further comprises the step of depositing graft and/or an implant at the joint.

In one embodiment, one or all of the components of the surgical system are disposable, peel-pack, pre-packed sterile devices. One or all of the components of the surgical system may be reusable. The surgical system may be configured as a kit with multiple sized and configured components.

In one embodiment, the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. In one embodiment, the present disclosure may be employed with other osteal and bone related applications, including those associated with diagnostics and therapeutics. In some embodiments, the disclosed surgical system and methods may be alternatively employed in a surgical treatment with a patient in a prone, supine position, lateral and/or employ various surgical approaches to the spine, including anterior, posterior, posterior mid-line, lateral, postero-lateral, and/or antero-lateral approaches, and in other body regions. The present disclosure may also be alternatively employed with procedures for treating the lumbar, cervical, thoracic, sacral and pelvic regions of a spinal column. The system and methods of the present disclosure may also be used on animals, bone models and other non-living substrates, such as, for example, in training, testing and demonstration.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, “treating” or “treatment” of a disease or condition refers to performing a procedure that may include administering one or more drugs to a patient (human, normal or otherwise or other mammal), employing implantable devices, and/or employing instruments that treat the disease, such as, for example, microdiscectomy instruments used to remove portions bulging or herniated discs and/or bone spurs, in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

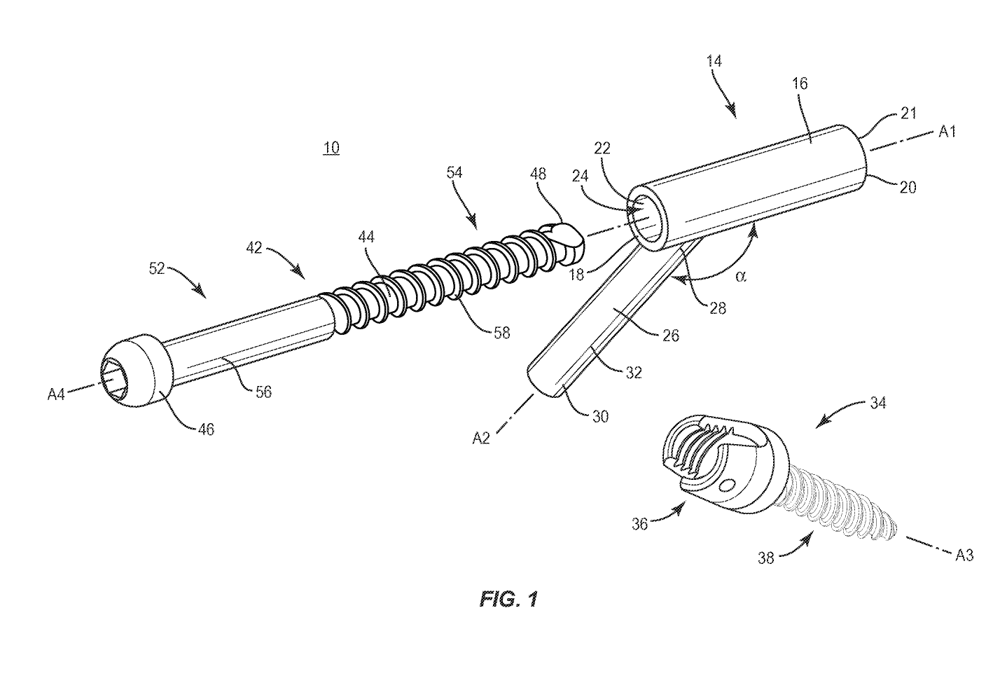

The following discussion includes a description of a surgical system and related methods of employing the surgical system in accordance with the principles of the present disclosure. Alternate embodiments are also disclosed. Reference will now be made in detail to the exemplary embodiments of the present disclosure, which are illustrated in the accompanying figures. Turning to FIG. 1, there are illustrated components of a surgical system, such as, for example, a spinal implant system 10.

The components of spinal implant system 10 can be fabricated from biologically acceptable materials suitable for medical applications, including metals, synthetic polymers, ceramics and bone material and/or their composites, depending on the particular application and/or preference of a medical practitioner. For example, the components of spinal implant system 10, individually or collectively, can be fabricated from materials such as stainless steel alloys, commercially pure titanium, titanium alloys, Grade 5 titanium, superelastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate such as hydroxyapatite (HA), corraline HA, biphasic calcium phosphate, tricalcium phosphate, or fluorapatite, tri-calcium phosphate (TOP), HA-TOP, calcium sulfate, or other resorbable polymers such as polyaetide, polyglycolide, polytyrosine carbonate, polycaroplaetohe and their combinations, biocompatible ceramics, mineralized collagen, bioactive glasses, porous metals, bone particles, bone fibers, morselized bone chips, bone morphogenetic proteins (BMP), such as BMP-2, BMP-4, BMP-7, rhBMP-2, or rhBMP-7, demineralized bone matrix (DBM), transforming growth factors (TGF, e.g., TGF-β), osteoblast cells, growth and differentiation factor (GDF), insulin-like growth factor 1, platelet-derived growth factor, fibroblast growth factor, or any combination thereof.

Various components of spinal implant system 10 may have material composites, including the above materials, to achieve various desired characteristics such as strength, rigidity, elasticity, compliance, biomechanical performance, durability and radiolucency or imaging preference. The components of spinal implant system 10, individually or collectively, may also be fabricated from a heterogeneous material such as a combination of two or more of the above-described materials. The components of spinal implant system 10 may be monolithically formed, integrally connected or include fastening elements and/or instruments, as described herein.

Spinal implant system 10 is employed, for example, with a minimally invasive procedure, including percutaneous techniques, mini-open and open surgical techniques to deliver and introduce instrumentation and/or an implant, such as, for example, a bone fastener, at a surgical site within a body of a patient, for example, a section of a spine. Spinal implant system 10 includes a spinal implant, such as, for example, a spinal construct 12 configured to facilitate drilling and/or tapping a cavity in vertebral tissue, guiding surgical instruments adjacent to a surgical site and/or delivering implants, such as, for example, bone fasteners to the surgical site.

Construct 12 includes a guide 14 comprising a first member, such as, for example a tube 16. Tube 16 axially extends between an end 18 and an end 20 and defines an axis A1. End 18 includes a planar end surface having a circumferential configuration and being disposed substantially perpendicular to axis A1. In some embodiments, the end surface of end 18 may be disposed at alternate orientations, relative to axis A1, such as, for example, transverse and/or other angular orientations, such as acute or obtuse, co-axial and/or may be offset or staggered.

End 20 includes a planar end surface having a circumferential configuration and disposed substantially perpendicular to axis A1. The planar end surface of end 20 defines a docking surface 21 configured to engage tissue, such as for example, vertebrae. In some embodiments, surface 21 may have alternate surface configurations, such as, for example, smooth, even, rough, threaded for connection with surgical instruments, arcuate, undulating, porous, semi-porous, dimpled, polished, friction and/or textured to enhance fixation with tissue. In some embodiments, surface 21 may be irregular, tapered, offset, staggered, uniform and non-uniform. Engagement of surface 21 with tissue can be flush, angled and/or spaced apart. In some embodiments, the end surface of end 20 may be disposed at alternate orientations, relative to axis A1, such as, for example, those alternatives described herein.

Tube 16 includes an inner surface 22 that defines a cavity, such as, for example, a guide channel 24. Channel 24 has a substantially linear configuration such that ends 18, 20 are substantially aligned. This configuration and alignment facilitates delivery and introduction of instrumentation and/or an implant to a surgical site. In some embodiments, tube 16 is configured as a drill and/or tap and/or screw guide and resists and/or prevents tissue damage, such as, for example, cervical nerve root damage and vertebral artery injury during implantation. In one embodiment, surface 22 may have alternate surface configurations, such as, for example, those alternative surface configurations described herein. In some embodiments, channel 24 is alternatively oriented relative to axis A1, such as, for example, transverse and/or other angular orientations, such as acute or obtuse, co-axial and/or may be offset or staggered, irregular, tapered, uniform and non-uniform.

Guide 14 includes a second member, such as, for example, a spinal rod 26 having a substantially linear configuration. Rod 26 defines an axis A2 extending in a transverse orientation relative to axis A1. In some embodiments, axis A2 can be disposed at a selected angular orientation α relative to axis A2. In one embodiment, angle α is greater than 90 degrees. In some embodiments, axis A2 may be disposed at alternative orientations relative to axis A1, such as, for example, those alternatives described herein.

Rod 26 extends between an end 28 and an end 30. End 28 is monolithically formed with tube 16 adjacent end 18 at a selected inclination angle α. Rod 26 includes an outer surface 32 and an end 30 configured for engagement with a fastener, discussed herein. In some embodiments, surface 32 may have alternate surface configurations, such as, for example, those alternatives described herein to enhance engagement with a fastener. In one embodiment, rod 26 is solid. In some embodiments, all or only a portion of rod 26 may be cannulated. In some embodiments, tube 16 may be integrally connected and/or fastened with rod 26.

System 10 includes a fastener, such as, for example, a bone screw 34. Screw 34 defines a longitudinal axis A3. In some embodiments, screw 34 may be engaged with tissue in various orientations, such as, for example, series, parallel, offset, staggered and/or alternate vertebral levels. In some embodiments, system 10 may include one or a plurality of screws 34. In some embodiments, screw 34 may comprise multi-axial screws, sagittal angulation screws, pedicle screws, mono-axial screws, uni-planar screws, fixed screws, tissue penetrating screws, conventional screws, expanding screws, wedges, anchors, buttons, clips, snaps, friction fittings, compressive fittings, expanding rivets, staples, nails, adhesives, posts, fixation plates and/or posts. In some embodiments, system 10 may comprise various instruments, such as, for example, inserters, extenders, reducers, spreaders, distractors, blades, retractors, clamps, forceps, elevators and drills, which may be alternately sized and dimensioned, and arranged as a kit.

Screw 34 comprises a first portion, such as, for example, a receiver 36 and a second portion, such as, for example, an elongated shaft 38 configured for penetrating tissue. Receiver 36 includes a pair of spaced apart arms having an inner surface that defines a U-shaped passageway. The passageway is configured for disposal of rod 26. In some embodiments, all or only a portion of the passageway may have alternate cross section configurations, such as, for example, oval, oblong, triangular, square, polygonal, irregular, uniform, non-uniform, offset, staggered, and/or tapered. In some embodiments, the arms of receiver 26 may be disposed at alternate orientations, relative to axis A3, such as, for example, those alternatives described herein.

In one embodiment, screw 34 has a multi axial configuration such that receiver 36 is rotatable to a selected angle through and within an angular range to capture rod 26 for fixation therein. The inner surface of receiver 36 includes a thread form configured for engagement with a coupling member, such as, for example, a set screw 40 (FIG. 5). Set screw 40 is threaded with receiver 36 to attach, provisionally fix and/or lock rod 26 with screw 34.

Shaft 38 has a cylindrical cross section configuration and includes an outer surface having an external thread form. In some embodiments, the external thread form may include a single thread turn or a plurality of discrete threads. In some embodiments, other engaging structures may be located on shaft 38, such as, for example, a nail configuration, barbs, expanding elements, raised elements and/or spikes to facilitate engagement of shaft 38 with tissue, such as, for example, vertebrae.

In some embodiments, all or only a portion of shaft 38 may have alternate cross section configurations, such as, for example, oval, oblong, triangular, square, polygonal, irregular, uniform, non-uniform, offset, staggered, undulating, arcuate, variable and/or tapered. In some embodiments, the outer surface of shaft 38 may include one or a plurality of openings. In some embodiments, all or only a portion of the outer surface of shaft 38 may have alternate surface configurations to enhance fixation with tissue, such as, for example, those alternatives described herein. In some embodiments, all or only a portion of shaft 38 may be disposed at alternate orientations, relative to axis A3, such as, for example, those alternatives described herein. In some embodiments, all or only a portion of shaft 38 may be cannulated.

System 10 includes a fastener, such as, for example a lag screw 42 that defines a longitudinal axis A4. Lag screw 42 is configured for disposal in channel 24. In some embodiments, lag screw 42 is employed to provide internal stabilization for vertebrae and to maintain orientation and position of the vertebrae as well as to provide stability to promote healing. Lag screw 42 includes a shaft 44 that extends between an end 46 and an end 48. End 46 includes a head 50 configured for engagement with a tool, such as, for example a driver. Head 50 includes an inner surface that defines a socket for receiving the tool. End 48 is configured for engagement with tissue, such as, for example, vertebrae. Shaft 44 includes a portion 52 and a portion 54. Portion 52 includes a substantially even surface 56. Portion 54 includes a threaded surface 58 for engaging vertebrae. In some embodiments, surface 56 and/or surface 58 may be alternately configured, such as, for example, those alternatives described herein.

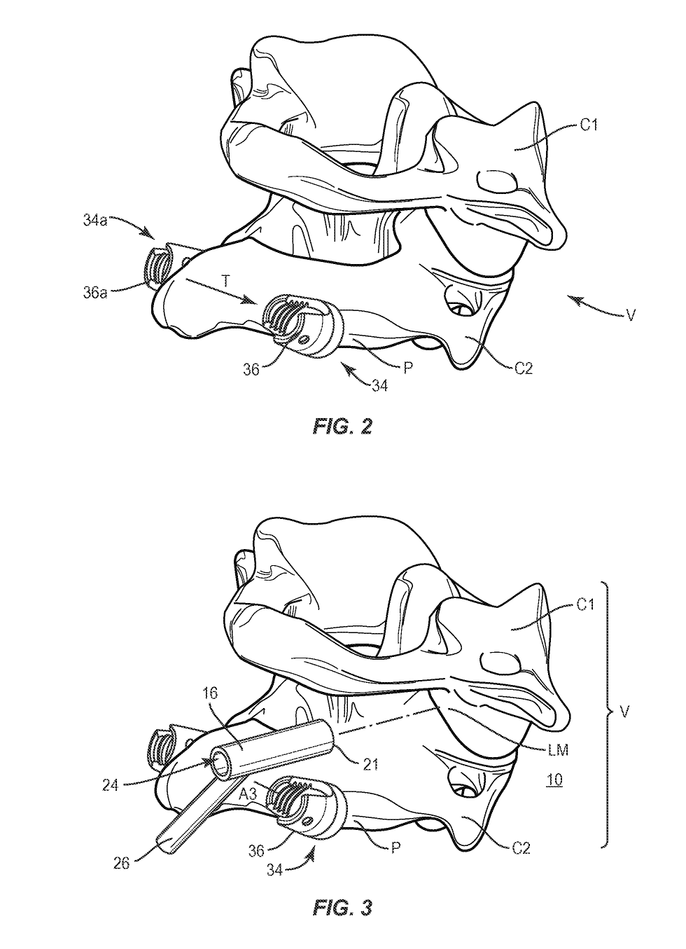

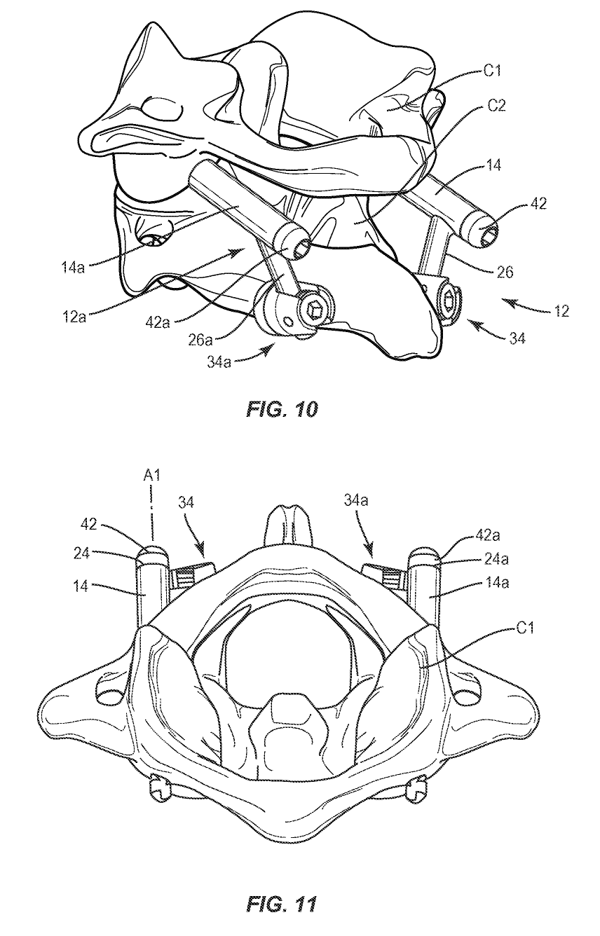

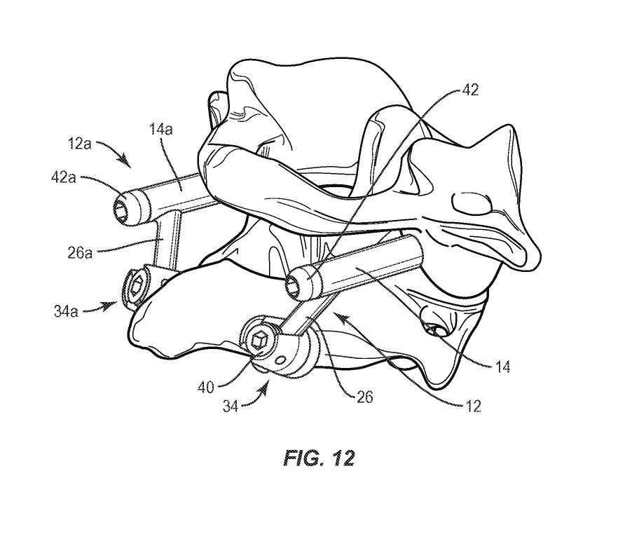

In assembly, operation and use, as shown in FIGS. 2-12, spinal implant system 10, similar to the systems described herein, is employed with a surgical procedure for treatment of a spinal disorder affecting a section of a spine of a patient, as discussed herein. System 10 may also be employed with other surgical procedures. For example, spinal implant system 10 can be used with a surgical procedure for treatment of a condition or injury of an affected cervical section of the spine including vertebrae.

In one embodiment, to treat the affected section of vertebrae V, a medical practitioner obtains access to a surgical site including the atlanto-axial joint in the upper part of the neck between the first and second cervical vertebrae C1, C2, as shown in FIG. 3, in any appropriate manner, such as through incision and retraction of tissues. In some embodiments, spinal implant system 10 may be used with any existing surgical method or technique including open surgery, mini-open surgery, minimally invasive surgery, and percutaneous surgical implantation, whereby vertebrae V is accessed through a micro-incision, or sleeve that provides a protected passageway to the area. Once access to the surgical site is obtained, the particular surgical procedure is performed for treating the spinal disorder. Spinal implant system 10 is then employed to augment the surgical treatment. Spinal implant system 10 can be delivered or implanted as a pre-assembled device or can be assembled in situ. Spinal implant system 10 may be completely or partially revised, removed or replaced, for example, removing tube 16 and/or rod 26, and/or one or all of the components of spinal implant system 10 before, during or after the surgical procedure.

A trajectory T is defined for insertion of screw 34 within a cervical section of a spine, as shown in FIG. 2. In one embodiment, trajectory T is determined using physical landmarks and fluoroscopy simultaneously. In one embodiment, trajectory T is parallel and in line with the appropriate joint leading up to the intersection of articular processes with the articular cartilage being the ending point. In some embodiments, screw 34 may be inserted via a trajectory oriented from an anterior, posterior, superior and/or inferior direction. An incision is made and fascia is cut to prepare for a dilator/delivery tube (not shown). In one embodiment, a guide wire and/or a trocar-cannula assembly may be employed as an instrument for gaining access to the surgical site and/or defining trajectory T.

A surgical passageway is created and the dilator/delivery tube may be employed to deliver screw 34 to the surgical site including vertebrae V, such as, for example, pars P of C2. In some embodiments, the dilator/delivery tube may be configured as an in-situ guidable instrument, and may include an endoscope camera tip for viewing insertion trajectory. Screw 34 is manipulable to drive, torque, insert or otherwise connect screw 34 with C2.

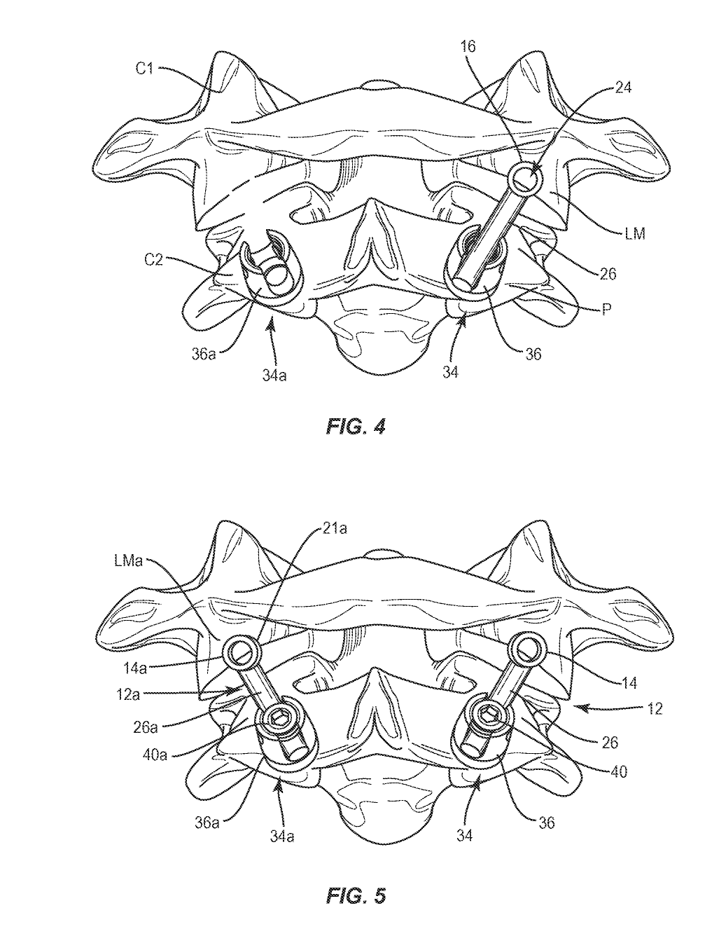

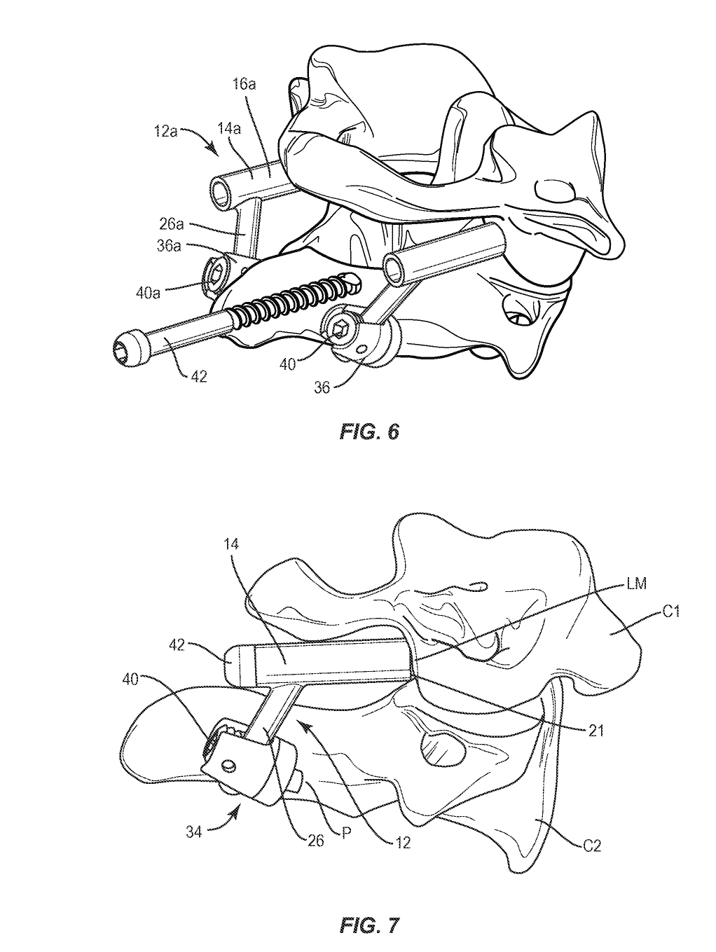

Guide 14 is delivered to the surgical site adjacent C1, C2, as shown in FIGS. 3 and 4. Guide 14 is aligned with a C1 lateral mass LM. Surface 21 is manipulated such that surface 21 is aligned with lateral mass LM and surface 21 engages with the surface of lateral mass LM as shown in FIG. 7. Positioning of tube 16 provides an access path to lateral mass LM through channel 24. Tube 16 is positioned such that rod 26 is docked in receiver 36 of screw 34, as shown in FIG. 4. Receiver 36 is manipulated to align the passageway of receiver 36 with the angle of rod 26 to capture rod 26 within receiver 36. Set screw 40 is engaged with receiver 36 to fix rod 26 in place. In some embodiments, tube 16 provides visual indicia along channel 24 of the trajectory of guide 14 with C1 and verification of selected alignment. Lateral mass LM can be drilled and/or tapped by inserting tools through channel 24 such that damage to the nerve root and/or vertebral artery is prevented. Lag screw 42 is inserted through channel 24 such that thread form 58 engages lateral mass LM for fixation of lag screw 42, guide 14 and spinal construct 12 with C1, C2, as shown in FIGS. 6-9.

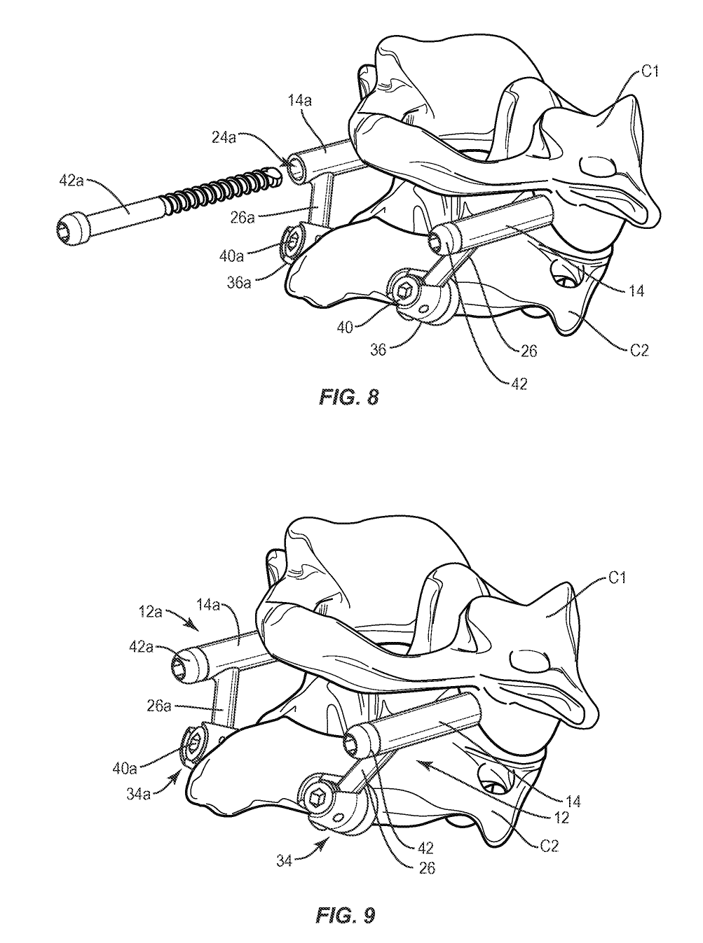

Spinal construct 12 is fixed to a lateral side of vertebrae C1, C2, as shown in FIGS. 7 and 8. In some embodiments, a spinal construct 12a, similar to spinal construct 12, is implanted on a contra-lateral side of C1, C2, as shown in FIGS. 8-12, such that spinal constructs 12, 12a are disposed in a bilateral configuration with C1, C2. A trajectory is defined for insertion of a screw 34a within a cervical section of a spine, similar to screw 34 described herein and shown in FIG. 2. Screw 34a is manipulable to drive, torque, insert or otherwise connect screw 34a with C2.

A guide 14a is delivered to the surgical site adjacent C1, C2, as shown in FIGS. 5 and 6. Guide 14a is aligned with a C1 lateral mass LMa, as shown in FIG. 5. A surface 21a is manipulated such that surface 21a is aligned with lateral mass LMa and surface 21a engages with the surface of lateral mass LMa. A tube 16a is positioned such that a rod 26a is docked in a receiver 36a of screw 34a. A set screw 40a is engaged with receiver 36a to fix rod 26a in place. Lateral mass LMa can be drilled and/or tapped by inserting tools through a channel 24a. A lag screw 42a is inserted through channel 24a for fixation of lag screw 42a, guide 14a and spinal construct 12a with C1, C2, as shown in FIGS. 8-12. Lag screws 42, 42a are disposed in parallel and the spinal constructs and fasteners are disposed away from the nerve root and/or vertebral artery adjacent C1, C2. In one some embodiments, bone graft can be inserted between the C1 arch and the C2 spinous process. In some embodiments, a connector and/or plate can be employed to link spinal constructs 12, 12a. In some embodiments, one or a plurality of spinal constructs can be disposed with one or a plurality of vertebra of vertebrae V.

In one embodiment, spinal implant system 10 includes an agent, which may be disposed, packed, coated or layered within, on or about the components and/or surfaces of spinal implant system 10. In some embodiments, the agent may include bone growth promoting material, such as, for example, bone graft to enhance fixation of the components and/or surfaces of spinal implant system 10 with vertebrae. In some embodiments, the agent may include one or a plurality of therapeutic agents and/or pharmacological agents for release, including sustained release, to treat, for example, pain, inflammation and degeneration.

Upon completion of the procedure, the surgical instruments, assemblies and non-implanted components of spinal implant system 10 are removed and the incision is closed. Spinal implant system 10 can be made of radiolucent materials such as polymers. Radiomarkers may be included for identification under x-ray, fluoroscopy, CT or other imaging techniques. In some embodiments, the use of surgical navigation, microsurgical and image guided technologies may be employed to access, view and repair spinal deterioration or damage, with the aid of spinal implant system 10. In some embodiments, spinal implant system 10 may include one or a plurality of plates, connectors and/or bone fasteners for use with a single vertebral level or a plurality of vertebral levels.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A spinal construct comprising:
a guide including a first member having an inner surface defining a guide cavity and a second member extending in a transverse orientation relative to the first member;
a first fastener including a first end defining an implant cavity and a second end configured for penetrating tissue; and
a second fastener disposable in the guide cavity such that the inner surface aligns the second fastener for penetrating tissue.

2. A spinal construct as recited in claim 1, wherein the first end is movable relative to the second end such the second member is disposable with the implant cavity.

3. A spinal construct as recited in claim 1, wherein the first end is rotatable relative to the second end in a configuration to selectively capture the second member within the implant cavity for fixation therewith.

4. A spinal construct as recited in claim 1, wherein first end is movable in a plurality of orientations relative to the second end.

5. A spinal construct as recited in claim 1, wherein the first fastener includes a multi-axial screw.

6. A spinal construct as recited in claim 1, wherein the first member defines a first axis and the second member defines a second axis, the first axis being disposed at a selected angular orientation relative to the second axis.

7. A spinal construct as recited in claim 1, wherein the first member defines a first axis and is attached with a first vertebra and the second member defines a second axis and is attached with a second vertebra such that the second axis is disposed at an angle of greater than substantially 90 degrees relative to the first axis.

8. A spinal construct as recited in claim 1, wherein the second fastener is configured to penetrate a C1 vertebra and the first fastener is configured to penetrate a C2 vertebra.

9. A spinal construct as recited in claim 1, wherein the guide cavity includes a linear channel.

10. A spinal construct as recited in claim 1, wherein the inner surface includes a substantially even surface.

11. A spinal construct as recited in claim 1, wherein the second fastener includes a first portion having a substantially even configuration and a second portion including a thread form.

12. A spinal construct as recited in claim 1, wherein the second fastener includes a lag screw configuration.

13. A spinal construct as recited in claim 1, wherein the first member includes a substantially tubular configuration and the second member includes a substantially solid configuration.

14. A spinal construct as recited in claim 1, wherein the first and second members are monolithically formed.

15. A spinal construct comprising:
a guide including a tube having a substantially even inner surface that defines a linear guide channel, the tube extending between a first end and a second end configured to engage a first vertebra, the guide further including a rod extending in a transverse orientation relative to the tube, the tube defining a first axis and the rod defining a second axis being disposed at an angular orientation relative to the first axis;
a first fastener including a first end defining an implant cavity and a second end configured for penetrating a second vertebra, the first end being movable in a plurality of axial orientations relative to the second end of the first fastener to selectively capture the rod within the implant cavity for fixation therewith; and
a second fastener disposable within the channel such that the inner surface aligns the second fastener for penetrating the first vertebra, the second fastener including a substantially even surface and a thread form.

16. A spinal construct comprising:
a first implant including a guide having a tube engageable with a lateral portion of a first vertebra and defining a guide channel and a rod extending in a transverse orientation relative to the tube, a multi-axial screw including an implant cavity configured for disposal of the rod and being attachable with a lateral portion of a second vertebra, and a lag screw disposable within the guide channel and attachable with the lateral portion of the first vertebra; and
a second implant including a guide having a tube engageable with a contra-lateral portion of the first vertebra and defining a guide channel and a rod extending in a transverse orientation relative to the tube of the second implant, a multi-axial screw including an implant cavity configured for disposal of the rod of the second implant and being attachable with a contra-lateral portion of the second vertebra, and a lag screw disposable within the guide channel of the second implant and attachable with the contra-lateral portion of the first vertebra.

17. A spinal construct as recited in claim 16, wherein the lag screw of the first implant and the lag screw of the second implant are disposed in a substantially parallel orientation.

18. A spinal construct as recited in claim 16, wherein the first and second implants are disposed in a bilateral configuration with the first vertebra and the second vertebra.

19. A spinal construct as recited in claim 16, wherein the first vertebra is a C1 vertebra and the second vertebra is a C2 vertebra.

20. A spinal construct as recited in claim 16, wherein each of the guide channels is configured for disposal of a tap for creating a cavity in the vertebrae, the cavities being spaced from nerve tissue of the vertebrae.
